(19) United States
(12) Patent Application Publication
(10) Pub. No.: US 2007/0031337 A1
(43) Pub. Date: Feb. 8, 2007

(54) Nanoparticle enhanced proton computed tomography and proton therapy

(76) Inventor: Reinhard Schulte, Grand Terrace, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/165,140

(22) Filed: Jun. 22, 2005

Related U.S. Application Data
(60) Provisional application No. 60/581,905, filed on Jun. 22, 2004.

(51) Int. Cl. A61N 5/00; A61K 49/00; A61K 33/24

(52) U.S. Cl. 424/9.6; 424/649; 977/906; 600/1

(57) ABSTRACT

Gold nanoparticles, which have a very high physical density, are bound to a specific antibody for cancer cells and then delivered to areas in which the tumors are believed to be present. The antigens of the cancer cells attract the antibodies bound to the gold nanoparticles so that the gold nanoparticles are bound to the cancer cells. With the increase of density caused by the gold nanoparticles, contrast between the cancer cells and the surrounding tissue is increased. Thus, the accuracy of detecting and characterizing tumors in a proton computed tomography system may be increased through the use of gold nanoparticles. Additionally, because the energy loss per path length of the protons after passing through the nanoparticles is larger than the energy loss per path length prior to reaching the nanoparticles, the nanoparticles may enhance the accuracy and increase radiation doses of current proton therapy systems.

### RELATED APPLICATIONS

This application claims priority under 35 U.S.C. § 119(e) to U.S. Provisional Application Ser. No. 60/581905, filed on Jun. 22, 2004, which is hereby expressly incorporated by reference in its entirety.

### FIELD OF THE INVENTION

The field of the invention relates to improved methods of radiation therapy and treatment planning.

### DESCRIPTION OF THE RELATED TECHNOLOGY

Conventional radiation therapy utilizes x-rays as a means of locating and treating tumors, such as cancer tumors. Due to the inability of conventional radiation treatment technology to preferentially deposit the radiation precisely at the site of the tumor, healthy tissues between the body surface and the tumor may also receive high doses of radiation and, thus, be damaged. Consequently, physicians may decide to use less-than-optimal doses in order to reduce the undesirable damage to healthy tissues and the subsequent side effects. Thus, there is a need for a radiation treatment system that accurately and reproducibly delivers the desired radiation treatment to designated target volumes with maximum sparing of dose-limiting healthy tissues.

In the recent past, proton therapy has emerged as a viable alternative to currently existing radiation treatment methods. While proton therapy has many principal advantages over conventional radiation therapy, systems and methods for more precise delivery of proton beams are desired to fully exploit these advantages.

Treatment planning, including tumor localization, normal tissue delineation and dose optimization, for proton therapy is commonly accomplished through the use of x-ray computed tomography (XCT) images. Accordingly, a patient undergoes XCT imaging, waits for an administering physician to develop a proton therapy treatment plan, and at some point in the future goes to a proton therapy treatment facility and is administered the developed treatment plan. In this embodiment, the patient is realigned on the treatment table in order to accurately administer the proton therapy. As those of skill and the art will appreciate, realigning a patient is a cumbersome process that often fails to realign the patient to the exact position that the patient was in when they XCT imaging was performed. In addition, changes in tumor size and its anatomic relationships would not be apparent at the time of treatment. Accordingly, systems and methods for implementing a proton therapy image guidance system are desirable.

### SUMMARY OF CERTAIN EMBODIMENTS

The systems, methods, and devices of the invention each have several aspects, no single one of which is solely responsible for its desirable attributes. Without limiting the scope of this invention, its more prominent features will now be discussed briefly. After considering this discussion, and particularly after reading the section entitled “Detailed Description of Certain Embodiments” one will understand how the features of this invention provide advantages over other display devices.

Proton radiation therapy is a precise form of radiation therapy. By offering greater precision than conventional radiation therapy, physicians are able to deliver higher, more effective doses to target volumes. Protons tend to travel through the body tissue without significant energy absorption until they reach a certain depth within the body, which depends on their initial energy. Beyond this depth, energy absorption increases significantly and abruptly falls to zero at the point where the protons stop. Because radiation dosage is directly related to energy absorption, proton radiation has a highest dose near the point where the protons stop.

Avoidance of damage to critical normal tissues and prevention of geographical tumor misses require accurate knowledge of the dose delivered to the patient and verification of the correct patient position with respect to the proton beam. In existing proton treatment centers, dose and proton range calculations are performed based on XCT and the patient is positioned with X-ray radiographs. However, the use of XCT images for proton treatment planning ignores fundamental differences in physical interaction processes between photons and protons and is, therefore, potentially inaccurate. Further, X-ray radiographs mainly depict patients' skeletal structures and rarely show the tumor itself. Accordingly, systems and methods for imaging patients directly with protons, for example, by measuring their energy loss after traversing the patients have recently been proposed. For example, Conceptual Design of a Proton Computed Tomography System for Applications in Proton Radiation Therapy, by Reinhard Schulte, Vladimir Bashkirov, Tianfang Li, Zhengrong Liang, Klaus Mueller, Jason Heimann, Leah R. Johnson, Brian Deeney, Hartmut F.-W. Sadrozinski, Abraham Seiden, David C. Williams, Lan Zhang, Zhang Li, Steven Peggs, Todd Satogata, and Craig Woody, 2003 IEEE NSS/MIC Portland, Oreg., which is hereby incorporated by reference in its entirety, describes exemplary systems and methods for use of proton CT in proton therapy treatment planning.

Conventional CT images, such as x-ray CT images, derive their tissue contrast from attenuation differences of photons as they pass through the body. This attenuation is proportional to the square of the average atomic number, Z, of the tissues traversed. Bones, consisting mainly of high-atomic calcium, may be relatively easy to distinguish from soft tissues. However, the composition of most tumors is very similar to normal soft tissues and distinguishing tumors from surrounding tissue may be difficult. In order to make tumors visible in XCT, a high-Z contrast material may be injected into the patient, which makes tumors more visible only if there is leakage of contrast material into the tumor tissue, which is not always the case. Moreover, this contrast material disturbs the dose calculation for a proton treatment plan and, therefore, limits its accuracy.

Using Proton Computed Tomography (pCT), it is possible to detect subtle differences in the density of the tissues on the beam path rather than in atomic number. Therefore, it more faithfully reproduces the physical characteristics of the tissues on the beam path and makes the proton treatment plan more accurate. However, the density difference between tumors and normal tissues may not be large enough to delineate the tumor without further density enhancement. As described in further detail below, in one embodiment gold nanoparticles, which have a very high physical density, are bound to a specific antibody for cancer cells and then delivered to areas in which the tumors are believed to be present. The antigens of the cancer cells attract the antibodies bound to the gold nanoparticles so that the gold nanoparticles are bound to the cancer cells. Accordingly, with the increase of density caused by the gold nanoparticles, contrast between the cancer cells and the surrounding tissue is increased. Moreover, tumor antibodies may be designed that are specifically directed to the cells of highest malignancy. Thus, the accuracy of detecting and characterizing tumors in a pCT system may be increased through the use of gold nanoparticles.

Currently, proton therapy is administered to patients in response to a treatment plan that was previously developed by XCT. Accordingly, the treatment that is provided to the patient is administered at a significantly later time and possibly in a different treatment position. As described in detail below, a system and method for providing image guided proton therapy provides real-time pCT images to an administering doctor or radiotherapist and allows immediate treatment planning and proton therapy based on the actual treatment position, anatomical configuration of a located tumor, and normal tissues that surround the tumor. Accordingly, the treatment may be more accurate than in conventional systems where treatment planning and the treatment itself are different events.

### BRIEF DESCRIPTION OF THE DRAWINGS

FIG. 1 is a schematic of an exemplary proton beam delivery system.

FIGS. 2A and 2B are exemplary schematics of antibody coated gold nanoparticles.

FIG. 3 is a diagram illustrating a plurality of antibody coated nanoparticles prepared for delivery to a tumor in a patient.

FIG. 4 is a diagram of a tumor with a plurality of antibody coated nanoparticles attached to an outer surface of the tumor.

FIG. 5 is a diagram illustrating the proton delivery module emitting a plurality of proton beams towards the patient.

FIG. 6 is a density enhancement chart illustrating the relationship of nanoparticle diameter and quantity to the degree of tumor density enhancement.

FIG. 7 chart illustrating simulated pCT scan data that was generated by the GEANT 4 Monte Carlo simulation code.

FIG. 8 depicts the reconstructed phantom image after delivery of simulated proton therapy.

FIG. 9 is a diagram illustrating the proton delivery module of FIG. 1 emitting a plurality of proton beams towards the tumor within the patient as part of a proton treatment plan.

### DETAILED DESCRIPTION OF CERTAIN EMBODIMENTS

In the following description, reference is made to the accompanying drawings, which form a part hereof, and which show, by way of illustration, specific examples or processes in which the invention may be practiced. Where possible, the same reference numbers are used throughout the drawings to refer to the same or like components. In some instances, numerous specific details are set forth in order to provide a thorough understanding of the invention. The invention, however, may be practiced without the specific details or with certain alternative equivalent devices and/or components and methods to those described herein. In other instances, well-known methods and devices and/or components have not been described in detail so as not to unnecessarily obscure aspects of the invention.

In one embodiment, proton CT can be use to generate models of the subject of interest, which may be viewed by the treatment planner and therapist in order to determine an appropriate proton therapy for immediate application. For example, a tumor may be located precisely while a patient is on the treatment table using proton CT, and immediately thereafter a proton therapy beam may be applied to the area identified using proton CT, where the proton therapy beam characteristics are determined by the proton CT images.

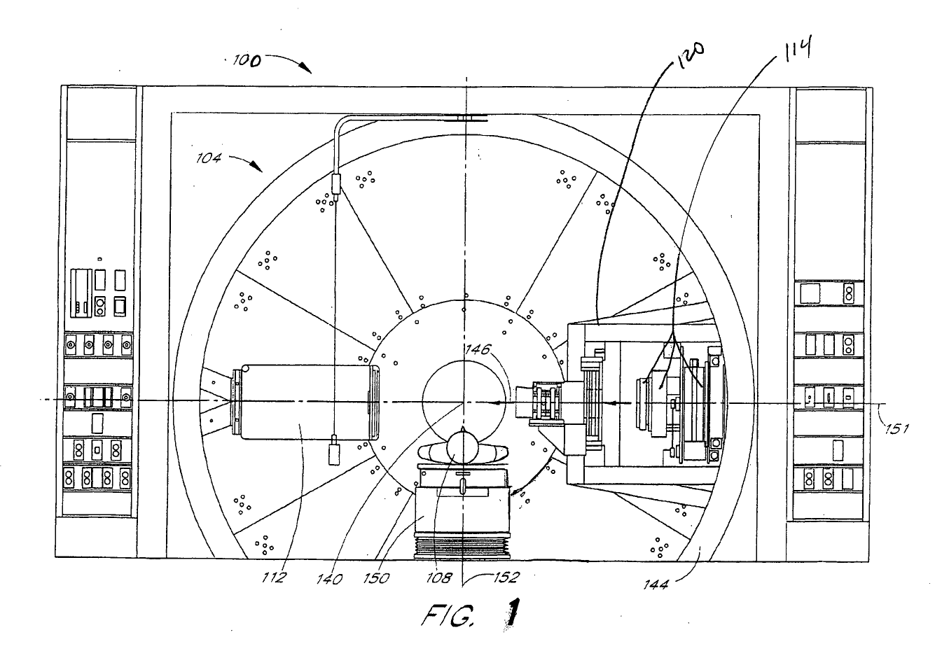

FIG. 1 is a schematic of an exemplary proton beam delivery system 100, including a gantry 104 that rotates about a center point (isocenter) 140. The exemplary beam delivery system 100 includes a proton beam delivery module 120, which includes beam-diagnostic and beam-modifying devices 114. A proton detection module 112 is mounted on the gantry at a position opposite the proton beam delivery module 120 so as to be centered about a beam path 146 that extends from the proton beam delivery module 120. Accordingly, the proton detection module 112 remains aligned with the proton beam delivery module 120 as the gantry 104 is rotated about the isocenter 140. As shown in FIG. 1, the gantry 104 is positioned so that the proton beam delivery module 120 emits a broad beam 146 centered on the beam axis 151. It will be appreciated, however, that the proton beam delivery module 120 can be rotated so that the beam axis 151 extends in a different direction but still intersects the isocenter 140. The beam delivery system 100 also includes a patient positioner 150 that is moveable along at least three orthogonal axes.

In the embodiment of FIG. 1, a patient 108 is positioned on top of the patient positioner 150. In one embodiment, the patient positioner 150 can be rotationally aligned. Other systems and methods of positioning patients are known and are contemplated for use in conjunction with the systems and methods described herein. For example, U.S. Pat. No. 4,905,267, titled “Method of Assembly and Whole Body, Patient Positioning and Repositioning Support for use in Radiation Beam Therapy Systems” and U.S. patent application Ser. No. 10/917023, titled “Patient Alignment System With External Measurement and Object Coordination for Radiation Therapy System,” which are hereby incorporated by reference in their entireties, describe other patient positioning systems and methods. In the embodiment of FIG. 1, when pCT and/or proton therapy is to be applied to the patient 108, the patient positioner 150 is moved so that an area of interest in the patient 108 is on the beam axis 151.

In one embodiment, the beam delivery system 100 is configured to provide pCT images for treatment planning, as well as administer a desired proton therapy. Accordingly, the beam delivery system 100 advantageously provides an image guided proton therapy system. In this embodiment, the proton beam delivery module 120 is configured to deliver (1) proton beams having an energy that is sufficient to pass through the patient 108 in order to be detected by the proton detection module 112 and (2) proton beams having energy that is calculated to provide a maximum radiation does to the determined target volume of the patient 108. Thus, the proton accelerator (not shown) generating protons to be transported to the proton beam delivery module 120 is configured to provide protons with various energy levels, depending on whether the beam delivery system 100 is developing PCT imagery or delivering proton beams to the patient 108.

In one embodiment, anti-bodies that are attracted by antigens of the tumor are coated with gold nanoparticles. There is currently much research being performed in determining tumor antigens, and their corresponding antibodies, that are present in cancerous tumors. In one embodiment, the antibodies are a few hundred nanometers wide, while the gold nanoparticles have diameters of a few nanometers to hundreds of nanometers. In addition, the relative sizes of the gold nanoparticles 210 and the antibodies 220 may be optimized according to the specific project needs.

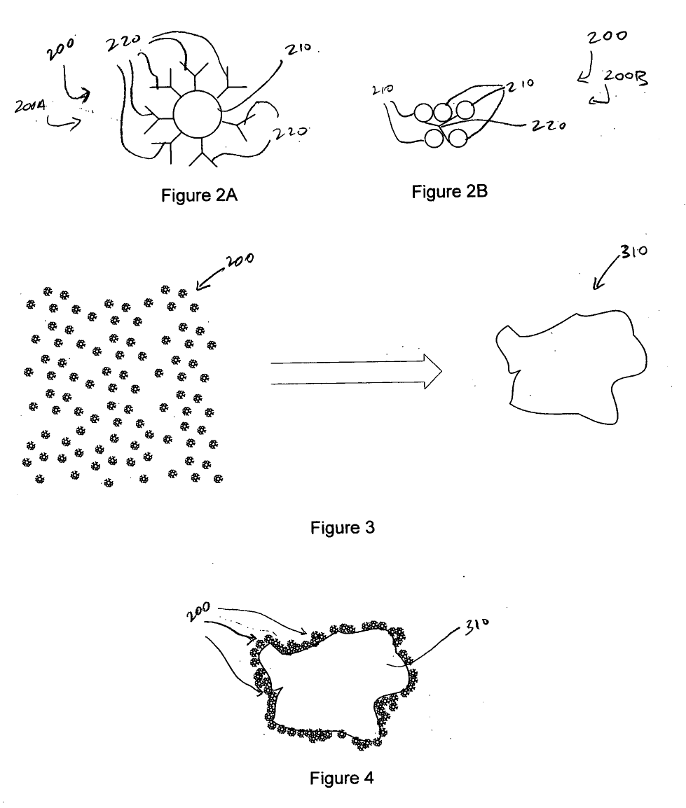

FIGS. 2A and 2B are exemplary schematics of antibody coated gold nanoparticles 200. In particular, the antibody coated nanoparticles 200A of FIG. 2A comprises a plurality of antibodies 220 conjugated to an outer surface of a single gold nanoparticle 210 and the antibody coated nanoparticle 200B comprises a plurality of gold nanoparticles 210 conjugated to a single antibody 220.

The gold nanoparticles 210 and antibodies 220 are not drawn to scale, but are illustrated schematically in order to demonstrate possible conjugations of gold nanoparticles with antibodies. In one embodiment, the antibodies are a few hundred nanometers wide, while the gold nanoparticles have diameters ranging from a few nanometers to a few hundred nanometers. Thus, in addition to the conjugations illustrated in FIGS. 2A and 2B, combinations of fewer or more antibodies 220 may be conjugated with fewer or more gold nanoparticles 210. References hereinafter to antibody coated nanoparticles 200 refer not only to the configurations illustrated in FIGS. 2A and 2B, but also to any other combination of antibodies and gold nanoparticles. For a discussion on exemplary gold nanoparticles, see, for example, Shape and Size Dependence of Radiative, Non-Radiative and Photothermal Properties Of Gold Nanocrystals, by Stephan Link and Mostafa A. El-Sayed, Annu. Rev. Phys. Chem., Vol. 19, No. 3, pp. 409-453 (2000), which is hereby incorporated by reference in its entirety.

Recent research, such as is discussed in Radiobiology For The Radiologist by Eric Hall, Lippincott-Raven, Philadelphia (2000), which is hereby incorporated by reference in its entirety, has indicated that a typical solid tumor contains about 10⁹ tumor cells. Other research has estimated that several hundred antibody coated nanoparticles can be attached to the surface of a tumor cell having antigens that match the antibodies conjugated to the gold nanoparticles. See, for example, Nanoshell-Mediated Near-Infrared Thermal Therapy of Tumors Under Magnetic Resonance Guidance, by L. R. Hirsch, R. J. Stafford, J. A. Bankson, S. R. Sershen, B. Rivera, R. E. Price, J. D. Hazle, N. J. Halas and J. L. West, Proc. Natl. Acad. Sci. USA Vol. 100, No. 23, pp. 13549-13554 (2003), which is hereby incorporated by reference in its entirety. Because not every cell of a tumor can be conjugated with an antibody, in one embodiment a typical solid tumor cell may carry in the range of about 50 to 500 antibody coated nanoparticles. In one embodiment, several thousand antibody coated nanoparticles are delivered to a large number of cells within a tumor site so that several hundred of the antibody coated nanoparticles attached to the surface of the tumor cell. As described in detail below, the attachment of gold nanoparticles 210 to the surface of a tumor may advantageously increase the ease of detecting the tumor, thereby providing more accurate and immediate pCT data, which may be immediately used to provide proton therapy to the tumor.

FIG. 3 is a diagram illustrating a plurality of antibody coated nanoparticles 200 prepared for delivery to a tumor cell 310 in a patient 108 (FIG. 1). As noted above, a tumor may comprises thousands to trillions or more tumor cells 310. While FIG. 3 illustrates only a single tumor cell 310, those of skill in the art will recognize that the antibody coated nanoparticles 200 may also be delivered to additional tumor cells throughout the tumor in the same manner. Antibody coated nanoparticles 200 may be delivered to the tumor cell 310 by any means currently known or hereafter developed, such as by intravenous injection or by direct injection into the tumor site of antibody coated nanoparticles 200

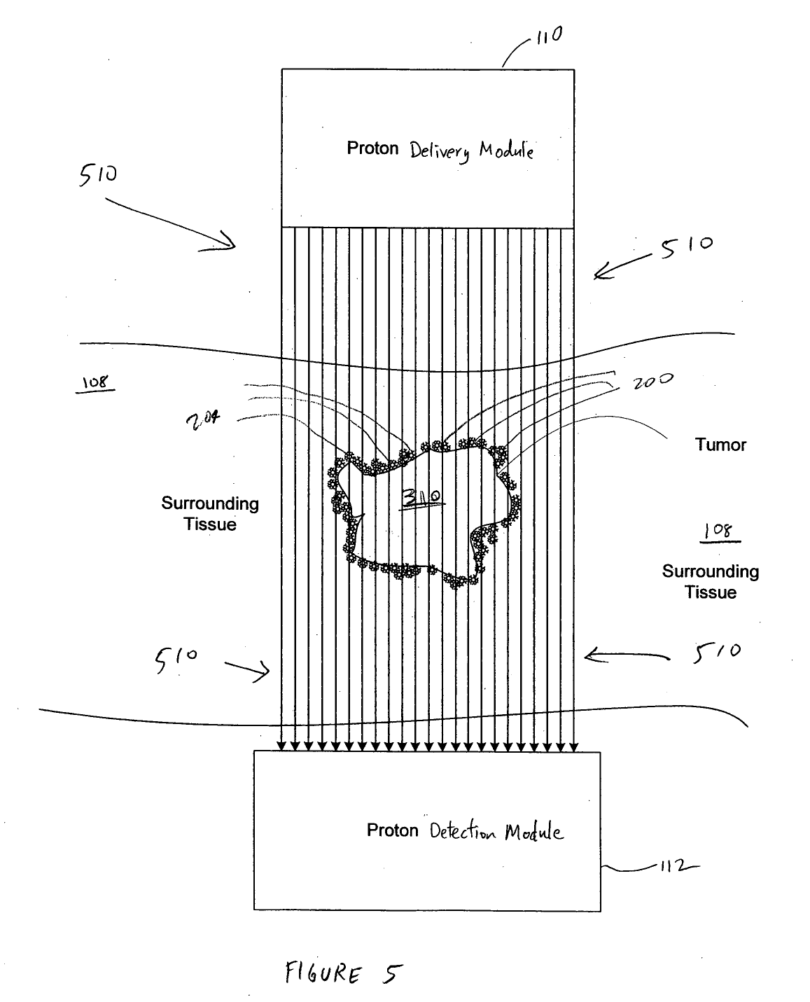

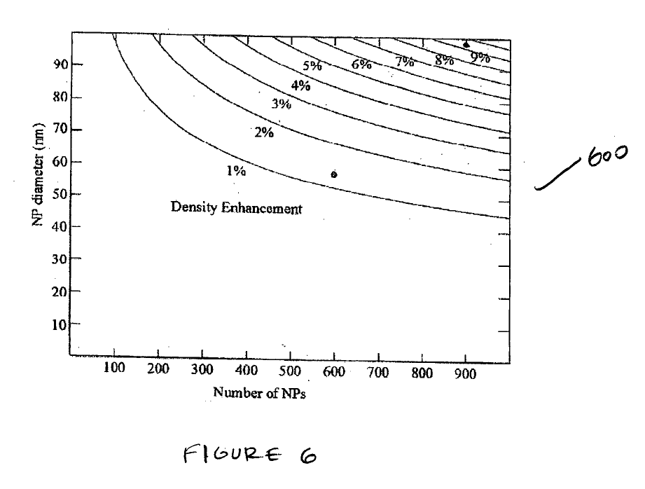

FIG. 4 is a diagram of the tumor cell 310 with a plurality of antibody coated nanoparticles 200 attached to the outer surface of the tumor cell 310. As noted above, depending on the size of the tumor, the tumor antigens, the antibody properties, the size of the gold nanoparticles, and the size of the antigens, among other factors, the number and size of the antibody coated nanoparticles 200 that attach to the tumor cell 310 may vary drastically.

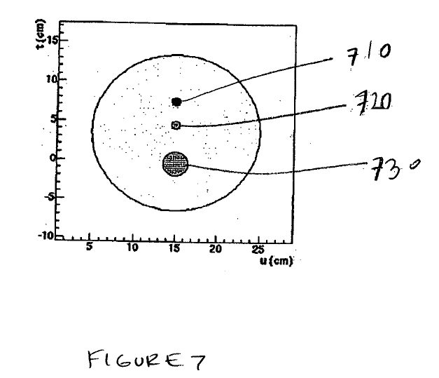

FIG. 5 is a diagram illustrating the proton beam delivery module 120 emitting a plurality of proton beams 510 towards the patient 108, and more specifically to an area of the patient suspected to contain a tumor. In FIG. 5, the proton beam delivery module 120 is configured to deliver proton beams 510 that traverse the tumor and the patient 108 so that an energy of each of the proton beams 510 may be detected by the proton detection module 112. Because higher energy protons are needed in order that the proton beams 510 emitted from the proton beam delivery module 120 pass completely through the subject of interest, such as the patient 108, in one embodiment the energy of the protons in the pCT step (FIG. 5) is greater than the energy of the protons in the treatment step (FIG. 6). In this way, the proton detection module 112 may develop 2D or 3D pCT images that may be immediately viewable by an administering radiotherapist. As described in further detail below with respect to FIG. 5, once a tumor, or other anomalies, are located in the patient 108, lower energy proton beams may be delivered by the proton beam delivery module 120 so that the maximum dose is delivered to the located tumor.

As illustrated in FIG. 5, a plurality of antibody coated nanoparticles 200 are advantageously attached to an outer surface of the tumor cell 310, and many other tumor cells throughout the tumor that are not shown in FIG. 5. In general, the antibody coated nanoparticles 200 reduce the energy of the proton beams so that the tumor cell 310 is more easily distinguishable from the surrounding tissue of the patient 108 when reconstructing tomographic images based on energy-loss measurements. More particularly, due to the high density of the gold nanoparticles that form a portion of each of the antibody coated nanoparticles 200, the energy of proton beams passing through the antibody coated nanoparticles 200 may be significantly decreased. Thus, the energy of those protons that have passed through the antibody coated nanoparticles 200 is less than the energy of those proton beams that do not pass through the antibody coated nanoparticles 200. Accordingly, the image area to which proton beams that pass through the plurality of tumor cells 310 contributed may be more easily detectable.

FIG. 6 is a density enhancement chart 600 illustrating the relationship of nanoparticle diameter, the number of nanoparticles per tumor cell 300, and the degree of tumor density enhancement. The figures illustrated in the density enhancement chart 600 assume a gold nanoparticle density of about 500 gold atoms per cubic nanometer. More particularly, the X-axis of the density enhancement chart 600 represents a diameter of the nanoparticles and the Y-axis represents a number of nanoparticles attached to a tumor cell. As illustrated in FIG. 6, with several hundred nanoparticles per tumor cell and nanoparticle diameters between about 60 and about 100 nm, density enhancements between about 1% and about 10% can be achieved. For example, a tumor with about 600 nanoparticles attached to its outer surface, where each nanoparticle has a diameter of about 60 nm, may exhibit about a 2% density enhancement. For a tumor having about 900 nanoparticles attached and a nanoparticle diameter of about 100 nm, a density enhancement of about 10% may be possible.

For a contrast enhancement of 1%, one needs to add about 10 mg gold or about 3×10¹⁸ gold atoms (atomic weight 196) to about 1 cm³ of tumor tissue, assuming unit density for the tumor tissue. With 10⁹ cells and 100 nanoparticles per cell this means that each nanoparticle should carry about 3×10⁸ gold atoms. In one embodiment, a gold nanoparticle of 10 nm diameter carries about 3×10⁵ gold atoms. In order to contain about 3×10⁸ gold atoms, the nanoparticles each have a diameter of about 100 nm.

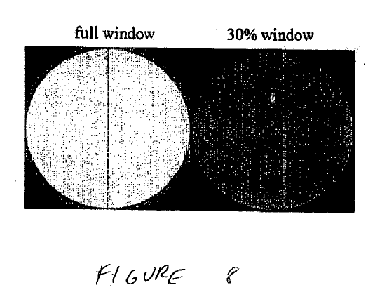

FIG. 7 chart schematically illustrates the cross section of a phantom used in a simulated pCT scan that was generated by the GEANT 4 Monte Carlo simulation code. More particularly, the GEANT4 simulation consisted of transport of a total of 6.3 million 200 MeV protons through a cylindrical water phantom of 20 cm diameter and 1 cm height with three gold enhanced water cylinders. As summarized in Table A, two of the cylinders 710 and 720 each had a diameter of 1 cm and respective densities of 1.127 g cm⁻³ and 1.013 g cm⁻³, while the third cylinder 730 had a diameter of 3 cm and a density of 1.013 g cm³. The phantom was centered at u=15 cm. The protons arrive along the u direction at plane u=0 cm. The proton detector is at u=30 cm.

TABLE A
Phantom Density Enhancements

| Gold enhanced | | | Diameter | Density | Density |
|---|---|---|---|---|---|
| water cylinder | u(cm) | t(cm) | (cm) | (g cm³) | enhancement |
| Cylinder 710 | 15 | 7.5 | 1.0 | 1.127 | 12.7% |
| Cylinder 720 | 15 | 4.5 | 1.0 | 1.013 | 1.3% |
| Cylinder 730 | 15 | −0.5 | 3.0 | 1.013 | 1.3% |

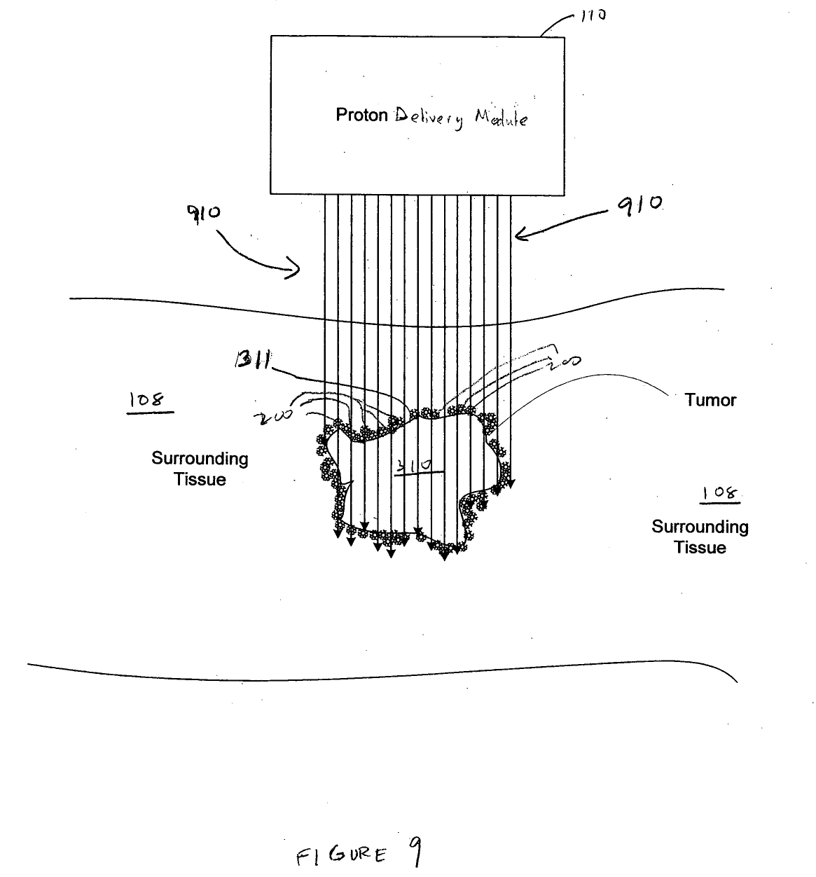

FIG. 8 depicts the reconstructed phantom image based on 6.3 million proton histories. More particularly, using GEANT 4, transport of a total of 6.3 million 200 MeV parallel, mono-energetic protons arriving at the plane u=0 cm with random vertical positions t, ranging from t=0 cm to t=7 cm, and being detected at the plane u=30 cm was simulated. The proton histories were equally distributed over 180 projections (0-360°, 35,000 protons per projection). The GEANT 4 simulation provided the location and direction of exiting protons as well as their residual energy. While the phantom 710 (FIG. 7) with a higher density of gold concentration and a corresponding 12.7% density enhancement is very well distinguished from the background water signal. However, the other two phantoms 720 and 730 (FIG. 7) with a lower density of gold concentration and a corresponding 1.2% density enhancements are only faintly visible. In one embodiment, it may be possible to increase the detectability of the low-density-enhanced regions by increasing the total number of protons. Thus, it is conceivable that the number of protons is adjusted to the degree of contrast difference expected between enhanced tumor tissue and normal tissue.

FIG. 9 is a diagram illustrating the proton beam delivery module 120 emitting a plurality of proton beams 910 towards the tumor cell 310 within the patient 108, and many other tumor cells throughout the tumor that are not shown in FIG. 9, as part of a proton therapy treatment. In an advantageous embodiment, the same proton beam delivery module 120 that was used to create the proton beams 510 and the pCT images of the patient 108 is also used for generation and delivery of the treatment proton beams 910. In this embodiment, the proton beam delivery module 120 is configurable so that the energy of the proton beams may be adjusted according to the data received from the pCT images. Advantageously, the patient 108 may remain positioned on the patient positioner 150 within the gantry 104 while pCT images are acquired and the proton treatment is administered. As those of skill in the art will understand, treatment may be much more accurate when imaging is provided concurrently with the treatment and guided by images produced by the same radiation source.

As illustrated in FIG. 9, the proton beams 910 are advantageously configured so that their energy is mostly released within the tumor cell 310 and not in front of or behind the tumor cell 310. Accordingly, the proton beams advantageously travel through the tumor cell 310, distributing a high dosage of energy after passing through the antibody coated nanoparticles 200 on the surface 311 of the tumor cell 310. In one embodiment, the energy loss per path length of the protons after passing through the antibody coated nanoparticles 200 is larger than the energy loss per path length prior to reaching the antibody coated nanoparticles 200 surrounding the tumor cell 310. In an advantageous embodiment, the energy of the proton beam is at an optimal level when it reaches the antibody coated nanoparticles 200 attached to the outer surface 311 of the tumor cell 310 such that the proton loses most or all of its residual energy within the tumor cell 310 or immediately outside the tumor. In one embodiment, the proton treatment plan dictates that most protons reach zero energy at different locations within the tumor so that portions of the tumor may receive substantially equal radiation or, alternative, so that portions of the tumor may receive more radiation than other portions of the tumor.

In one embodiment, the energy loss per path length is proportional to the density of the material the proton beam is passing through. Accordingly, the energy loss per path length is proportional to the Z (atomic number) of the material. Thus, for a high Z material, the energy loss per path length will increase. The energy loss per path length is proportional to dose and, thus, when the energy loss per path length increases the dose also increases. Because the energy loss per path length increases after a proton beam passes through a gold nanoparticle, the dose supplied by the proton beam after passing through the antibody coated nanoparticle 200 increases. In this way, a given dose may be supplied to a tumor with fewer protons than without the use of gold nanoparticles, or alternatively, a higher dose may be delivered to the tumor for the same amount of dose to the surrounding tissues. While the use of gold nanoparticles has been described in detail above, it is expressly contemplated that other high-Z materials, alone or in combination, may also be conjugated with antibodies, or other tumor seeking materials, for use with the systems and methods described herein. In addition, other markers or marker materials, whether nanoparticles, larger particles, liquids, or gases may be coupled to tumor cells in order to increase recognition of tumors through pCT and/or increase efficiency of proton therapy.

Specific parts, shapes, materials, functions and modules have been set forth, herein. However, a skilled technologist will realize that there are many ways to fabricate the system of the present invention, and that there are many parts, components, modules or functions that may be substituted for those listed above. While the above detailed description has shown, described, and pointed out the fundamental novel features of the invention as applied to various embodiments, it will be understood that various omissions and substitutions and changes in the form and details of the components illustrated may be made by those skilled in the art, without departing from the spirit or essential characteristics of the invention.

What is claimed is:

1. An image guided proton therapy method comprising:
delivering a plurality of nanoparticles to a tumor comprising a plurality of tumor cells so that at least some of the nanoparticles are coupled to at least some of the tumor cells;
transmitting a proton beam through at least a portion of the tumor;
measuring an energy loss of at least a portion of the proton beam after passing through the at least a portion of the tumor;
determining a treatment of the tumor based upon the step of measuring energy loss;
transmitting a treatment proton beam through the at least a portion of the tumor in response to the determined treatment.

2. The method of claim 1, wherein the nanoparticles comprise gold.

3. The method of claim 2, wherein the gold nanoparticles each have a density of about 5×10² gold atoms per cubic nanometer.

4. The method of claim 2, wherein the gold nanoparticles each have a diameter in the range of about 60 to 100 nanometers.

5. A proton computed tomography method comprising:
transmitting a proton beam through at least a portion of a tumor and tissue surrounding the tumor, the tumor having a marker material attached to an outer surface of the tumor;
measuring an energy loss of at least a portion of the proton beam after passing through the at least a portion of the tumor, wherein the marker material is configured to enhance contrast of the tumor from materials surrounding the tumor;
generating images representative of the tumor and the tissue surrounding the tumore, wherein the images are generated at least partly based on the measured energy loss.

6. The method of claim 5, wherein the marker material comprises a plurality of nanoparticles.

7. The method of claim 6, wherein the marker material comprises a plurality of gold nanoparticles.

8. The method of claim 7, wherein the marker material comprises a plurality of gold nanoparticles conjugated with a plurality of antibodies.

9. The method of claim 6, wherein the marker material comprises a plurality of high-Z nanoparticles.

10. The method of claim 5, wherein the images comprises 3D images of a human in which the tumor is disposed.

11. The method of claim 5, wherein the proton beam comprises a plurality of protons.

12. The method of claim 5, further comprising:
determining a treatment of the tumor in response to the measuring.

13. A method of improving proton radiation treatment planning, comprising:
delivering antibody-coated markers to a targeted object;
irradiating the targeted object with a plurality of protons;
tracking the path of the plurality of protons;
measuring the energy loss of at least a subset of the plurality of protons; and
forming proton computed tomography images based on the measured energy loss of the at least a subset of the plurality of protons.

14. The method of claim 13, wherein the marker material comprises gold nanoparticles.

15. A tumor location and treatment system comprising:
a target volume comprising a tumor, wherein a plurality of nanoparticles are attached to the tumor;
a proton delivery module configured to generate protons for selectively irradiating the target volume; and
a proton detection module configured to detect protons from the proton delivery module, the target volume being positioned between the proton delivery module and the proton detection module;
wherein, in a first mode the proton delivery module is configured to generate protons that traverse the target volume and reach the proton detection module, the proton detection module being configured to detect the tumor in the target volume based on energy levels of the protons reaching the proton detection module, and in a second mode the proton delivery module is configured to generate protons with energy levels sufficient to traverse a portion of the target volume and then lose their energy in the tumor that is detected.

16. The system of claim 15, wherein the target volume comprises a portion of a human.

17. The system of claim 15, wherein the nanoparticles comprise gold.

18. The system of claim 17, wherein at least some of the nanoparticles comprise in the range of about 3×10⁷ to 3×10⁹ gold atoms.

19. The system of claim 15, wherein the nanoparticles comprise a high-Z material.

20. The system of claim 15, wherein nanoparticles comprise a material that has an affinity to the tumor.

21. The system of claim 15, wherein the energy level of the protons decreases as the protons pass through the nanoparticles.

22. The system of claim 21, wherein the nanoparticles increase a difference in the energy levels of the protons that traverse the tumor and the protons that only traverse the target volume surrounding the tumor.

23. The system of claim 15, wherein the tumor comprises about 10⁹ tumor cells.

24. The system of claim 15, wherein each of the nanoparticles comprises at least one antibody having an affinity for the tumor.

25. The system of claim 15, wherein the energy of the protons generated in the first mode is in the range of about 100 to 300 MeV.

26. The system of claim 15, wherein the energy of the protons generated in the second mode is in the range of about 10 to 300 MeV.

27. A proton radiation treatment planning system comprising:
means for delivering antibody-coated gold nanoparticles to a targeted object;
means for irradiating the targeted object with a plurality of protons;
means for tracking the path of the plurality of protons;
means for measuring the energy loss of at least a subset of the plurality of protons; and
means for forming proton computed tomography images based on the measured energy loss of the at least a subset of the plurality of protons.
